SEQUENCE LISTING (18 sequences)

SEQ ID NO: 1 - DNA, 22 bp, Homo sapiens
  Artificial Sequence (1)..(22): KRT10-F
tcctacttggacaaagttcggg

SEQ ID NO: 2 - DNA, 19 bp, Homo sapiens
  Artificial Sequence (1)..(19): KRT10-R
cccctgatgtgagttgcca

SEQ ID NO: 3 - DNA, 21 bp, Homo sapiens
  Artificial Sequence (1)..(21): KRT1-F
agagtggaccaactgaagagt

SEQ ID NO: 4 - DNA, 21 bp, Homo sapiens
  Artificial Sequence (1)..(21): KRT1-R
attctctgcatttgtccgctt

SEQ ID NO: 5 - DNA, 20 bp, Homo sapiens
  Artificial Sequence (1)..(20): SMPD1-F
ctgactctcgggttctctgg

SEQ ID NO: 6 - DNA, 20 bp, Homo sapiens
  Artificial Sequence (1)..(20): SMPD1-R
tccaccatgtcatcctcaaa

SEQ ID NO: 7 - DNA, 20 bp, Homo sapiens
  Artificial Sequence (1)..(20): TBP-F
tataatcccaagcggtttgc

SEQ ID NO: 8 - DNA, 20 bp, Homo sapiens
  Artificial Sequence (1)..(20): TBP-R
gctggaaaacccaacttctg

SEQ ID NO: 9 - DNA, 22 bp, Homo sapiens
  Artificial Sequence (1)..(22): TYRP1-F
gacacgcctcctttttattcca

SEQ ID NO: 10 - DNA, 20 bp, Homo sapiens
  Artificial Sequence (1)..(20): TYRP1-R
atgggtttgtccccctgttc

SEQ ID NO: 11 - DNA, 20 bp, Homo sapiens
  Artificial Sequence (1)..(20): TYR-F
ctcaaagcagcatgcacaat

SEQ ID NO: 12 - DNA, 20 bp, Homo sapiens
  Artificial Sequence (1)..(20): TYR-R
gcccagatctttggatgaaa

SEQ ID NO: 13 - DNA, 20 bp, Homo sapiens
  Artificial Sequence (1)..(20): MITF-F
gcctccaagcctccgataag

SEQ ID NO: 14 - DNA, 21 bp, Homo sapiens
  Artificial Sequence (1)..(21): MITF-R
gcactctctgttgcatgaact

SEQ ID NO: 15 - DNA, 22 bp, Homo sapiens
  Artificial Sequence (1)..(22): MC1R-F
catcatcgaccccctcatctac

SEQ ID NO: 16 - DNA, 24 bp, Homo sapiens
  Artificial Sequence (1)..(24): MC1R-R
caggaaccagaccacacaatatca

SEQ ID NO: 17 - DNA, 19 bp, Homo sapiens
  Artificial Sequence (1)..(19): GAPDH-F
ctgggctacactgagcacc

SEQ ID NO: 18 - DNA, 21 bp, Homo sapiens
  Artificial Sequence (1)..(21): GAPDH-R
aagtggtcgttgagggcaatg

(19) United States
(12) Patent Application Publication
(10) Pub. No.: US 2022/0054398 A1
(43) Pub. Date: Feb. 24, 2022

(54) USE OF RUBUS FRUTICOSUS EXTRACT FOR MANUFACTURING A SKINCARE COMPOSITION

(71) Applicant: TCI CO., LTD., Taipei (TW)

(72) Inventors: YUNG-HSIANG LIN, Taipei (TW); WEI-CHUN LEE, Taipei (TW)

(21) Appl. No.: 17/407,190

(22) Filed: Aug. 20, 2021

Related U.S. Application Data
(60) Provisional application No. 63/068,396, filed on Aug. 21, 2020.

(30) Foreign Application Priority Data
Jul. 6, 2021 (TW) 110124861

(51) Int. Cl. A61K 8/9789; A61Q 19/02; A61Q 19/08; A61Q 19/00; A23L 33/105

(52) U.S. Cl. CPC A61K 8/9789; A61Q 19/02; A61K 2800/805; A61Q 19/007; A23L 33/105; A61Q 19/08

(57) ABSTRACT

The present disclosure provides a use of Rubus fruticosus extract for manufacturing a skincare composition, where the composition is used for reducing oxidative damage caused by reactive oxygen species in cells, reducing the amount of hemoglobin in skin, inhibiting melanin production, reducing skin redness spots, and enhance the skin's moisturizing ability.

### CROSS-REFERENCES TO RELATED APPLICATIONS

This application claims the benefit of U.S. provisional application Ser. No. 63/068,396, filed on Aug. 21, 2020 and claims the priority of Patent Application No. 110124861 filed in Taiwan, R.O.C. on Jul. 6, 2021. The entirety of the above-mentioned patent application is hereby incorporated by reference herein and made a part of the specification.

### REFERENCE OF AN ELECTRONIC SEQUENCE LISTING

The contents of the electronic sequence listing (P201032 USI_ST25.txt; Size: 4.25 KB; and Date of Creation: Aug. 20, 2021) is herein incorporated by reference in its entirety.

### BACKGROUND OF THE INVENTION

### 1. Field of the Invention

The present disclosure relates to a Rubus fruticosus extract, and a method of manufacturing skin conditioning compositions with Rubus fruticosus extract.

### 2. The Prior Art

Along with environmental changes, ultraviolet (UV) index and air pollution becomes more and more serious. UV radiation of the sun is the main cause of skin damage, aging, tanning. Skin is exposed to the sun for a long time will produce melanin and form spots, which is difficult to be eliminated. In addition, UV radiation in the sun will promote the production of reactive oxygen species (ROS, also known as free radicals) in skin cells, resulting in skin aging, loss of brightness, dryness, and redness.

In addition, as the skin cells gradually aging, and the skin texture becomes rough and forms wrinkles. Because metabolism of skin cells is slowing down, the skin's recovery speed is also gradually slowing down. In addition, long-term exposure to external environmental stimuli, such as air pollution, accumulates a large amount of active oxygen substances and melanin that cannot be completely metabolized in time. As a result, the skin condition cannot be improved.

Most of the known cosmetics, skincare products and health products are made of chemical ingredients, which are harmful to human health, and irritates sensitive skin and aggravates skin redness. In addition, the manufacturing process is not friendly to the environment and ecology, causing environmental pollution, and even entering the ecosystem through the drainage system.

In order to solve these problems, there is an urgent need for scientists in this field to develop skin conditioning compounds containing anti-UV, anti-oxidant, melanin inhibiting, redness reducing, redness relieving, and skin moisturizing capacity for the benefit of the general population.

### SUMMARY OF THE INVENTION

An objective of the present disclosure is to provide a method of manufacturing a skin conditioning composition with Rubus fruticosus extract. The composition is used for inhibiting melanin production and reducing skin redness by reducing the amount of melanin-related genes expression in cells. The composition is used for reducing oxidative damage caused by reactive oxygen species in cells to achieve an antioxidant effect, protect skin cells, achieve antioxidant, skin lightening, and reduce skin redness.

Another objective of the present disclosure is to provide a method of manufacturing a skin conditioning composition with Rubus fruticosus extract to reduce erythema index in skin, relieve the skin's redness, and improve the skin's moisturizing capacity, increase the skin's brightness, and slow down skin aging.

A method of manufacturing a skin conditioning composition with Rubus fruticosus extract, wherein the Rubus fruticosus extract is extracted with a solvent containing water.

In some embodiments, a total polyphenol content of the Rubus fruticosus extract ranges from 8000 and 11000 ppm.

In some embodiments, the composition is for reducing oxidative damage caused by reactive oxygen species in cells.

In some embodiments, the composition is for reducing a hematocrit level of the skin.

In some embodiments, the composition is for inhibiting melanin production.

In some embodiments, the composition inhibits melanin production by reducing an amount of melanin-related genes expressions in cells.

In some embodiments, the melanin-related genes includes tyrosinase-related protein-1 (TYRP1), tyrosinase (TYR), microphthalmia-associated transcription factor (MITF), and melanocortin receptor 1 (MC1R).

In some embodiments, the composition is for reducing skin redness.

In some embodiments, the composition is for enhancing a moisturizing ability of the skin.

In some embodiments, the composition enhances moisturizing ability of the skin by increasing an amount of moisturization-related genes expressions in the cells.

In some embodiments, the composition is for manufacturing a nutraceutical composition, a health product composition, a cosmetic composition, or a topical skin application.

In summary, the Rubus fruticosus extract of any embodiment inhibits the production of reactive oxygen species, inhibits melanin production by reducing the expression of melanin-related genes (e.g., tyrosinase-related protein-1 (TYRP1), tyrosinase (TYR), microphthalmia-associated transcription factor (MITF), melanocortin receptor 1 (MC1R)) in cells. Therefore, the Rubus fruticosus extract is effective for preventing skin aging phenomena caused by UV rays, achieving anti-oxidation, whitening, and reducing skin redness. The Rubus fruticosus extract provides skin protection against UV rays and blue light.

In addition, the Rubus fruticosus extract reduces erythema index of skin, relieves skin redness, improves the skin's moisturizing capacity, increasing skin brightness.

### BRIEF DESCRIPTION OF THE DRAWINGS

FIG. 1 is a comparison chart of the results of proportions of cells highly expresses reactive oxygen species (ROS) in a control group, a comparison group, and an experimental group. ###p value<0.001, compared with the control group, *** p value<0.001, compared with the comparison group.

FIG. 2 is a comparison chart of results of expression levels of moisturizing-related genes in the control group and the experimental group. ***p value<0.001, compared to the control group.

FIG. 3 is a comparison chart of the results of the expression levels of melanin-related genes in the control group and the experimental group. **p value<0.01, ***p value<0.001, compared to the control group.

FIG. 4 is a comparison chart of the results of the melanin content of the control group, the control group, and the experimental group. ***p value<0.001, compared to the control group.

FIGS. 5A and 5B are a comparison chart of results of the number of ultraviolet red spots on the skin of subjects and the images taken by the detector after the subjects continuously intake beverages containing the experimental group at the 0th week, the 4th week, and the 8th week, and the images taken by a detector. The subject's UV red spots before intake is 100%. *p value<0.1, compared to 0th week.

FIGS. 6A and 6B show the skin erythema index of the subjects and the images taken by the detector after the subjects continuously intake the beverages containing the experimental group at the 0th week, the 4th week, and the 8th week. The subject's skin erythema index before intake is 100%. *p value<0.1, ***p value<0.001, compared to 0th week.

FIGS. 7A and 7B show the changes of skin brightness and the images taken by the detector after the subjects continuously intake the drink containing the experimental group at the 0th week, the 4th week, and the 8th week. The brightness of the subjects is 100%. *p value<0.1, compared to 0th week.

### DETAILED DESCRIPTION OF THE PREFERRED EMBODIMENT

In the following detailed description of some embodiments of the present disclosure in which the present disclosure may be practiced. It is understood that other embodiments may be used and that changes can be made to the embodiments without departing from the scope of the present disclosure. The following description is therefore not to be considered as limiting the scope of the present disclosure.

Statistical analysis is performed using Excel. Data are expressed as mean±standard deviation (SD), and the difference between each group is analyzed by the Student's t-test.

As used herein, the data provided represent experimental values that can vary within a range of ±10%, and most preferably within ±5%.

As used herein, the term “extract” refers to a product prepared through extraction. The extract can be presented in the form of a liquid solution or a form of a concentrate or essence containing no or little solvent.

The Rubus fruticosus (also known as blackberry or dewberry) is the fruit of a shrub in the genus Rubus in the rose family.

As used herein, “Rubus fruticosus” generally means a plant fruit. The fruit may include fresh, dried, or being processed by other physical methods. The physical methods may include whole, chopping, dicing, milling, or grounding. Any physical process that renders the seed different from its original physical property is considered a physical method.

In some embodiments, the “Rubus fruticosus extract” may be the juice obtained by squeezing the fruit of the Rubus fruticosus. For example, in some embodiments, the fruit of the Rubus fruticosus is crushed, squeezed, and then concentrated to a sugar level after the pomace and smaller suspensions have been removed. In other embodiments, the Rubus fruticosus extract may also be subjected to other processes that do not substantially affect its ability to produce the effects described herein during preparation, such as fermentation, in order to add flavor or other uses.

In some embodiments, the “Rubus fruticosus extract” may also be an extract obtained by extracting the fruit of the Rubus fruticosus using a suitable extracting solvent. For example, the Rubus fruticosus may be extracted by soaking in water at room temperature for a suitable period of time, and then filtered to remove solid impurities to obtain a Rubus fruticosus extract.

In some embodiments, the juice extracted from the Rubus fruticosus (sometimes referred to as “Rubus fruticosus extract”) inhibits free radical production, reduces the erythema index of the skin, inhibits melanin production, reduces the amount of redness in the skin, and enhances the moisturizing ability of the skin. Therefore, Rubus fruticosus extract can be used for manufacturing skin conditioning composition that inhibits free radical production, reduces skin's erythema index, inhibits melanin production, reduces the number of red spots on the skin, and enhances the skin's moisturizing ability.

In some embodiments, the aforementioned composition may be a pharmaceutical composition, a cosmeceutical composition, a food composition, and a health product composition.

A pharmaceutical compound may be manufactured into a dosage form suitable for enterally, parenterally, or topically delivery using techniques well known to people skilled in the art. Examples may include injection (e.g. sterile aqueous solution or dispersion), sterile powder, external formulation, or other similar substances.

The pharmaceutical composition may further contain a widely used pharmaceutically acceptable carrier. For example, the pharmaceutically acceptable carrier contains one or more of the following reagents: emulsifiers, suspending agents, decomposers, disintegrating agents, dispersing agents, binding agents, excipients, stabilizers, chelating agents, diluents, gelling agents, preservatives, wetting agents, lubricants, absorption delaying agents, liposomes, and the same. The selection and quantification of these agents are routine practice and techniques for people having ordinary skill in the art.

For example, the pharmaceutical composition may be provided in a dosage form suitable for oral administration by any suitable method, wherein the liquid dosage form suitable for oral administration includes syrup, oral liquid, suspension, elixir, etc. The solid dosage form suitable for oral administration includes powder, granule, oral tablet, sugar-coated tablet, enteric tablet, chewable tablet, effervescent tablet, film-coated tablet, capsule, long-acting slow-release tablet, etc. The pharmaceutical composition according to the present invention may contain any pharmaceutically acceptable carrier that does not adversely affect the desired benefit of the active ingredient (i.e., Bifidobacterium lactis TCI604 and/or its metabolites). For example, but is not limited to, examples of the aforementioned pharmaceutically acceptable carriers for liquid dosage forms include: water, saline, glucose (dextrose), glycerol, ethanol, or its analogues, oils (e.g., olive oil, castor oil, cottonseed oil, peanut oil, corn oil, and germ oil), glycerin, polyethylene glycol, and combinations thereof. Examples of the aforementioned pharmaceutically acceptable carriers for solid dosage forms include cellulose, starch, kaolinite, bentonite, sodium citrate, gelatin, agar, carboxymethylcellulose, arabic gum, algae gum, glyceryl monostearate, calcium stearate, and combinations thereof.

In some embodiments, the pharmaceutically acceptable carrier agent may comprise one of the following solvents: water, normal saline, phosphate buffered saline (PBS), aqueous solution containing alcohol, and any other suitable solvents.

In some embodiments, the pharmaceutical compound may be administered by any of the following non-parenteral routes: subcutaneous injection, intraepidermal injection, intradermal injection, intralesional injection, and intralesional injection.

In some embodiments, the pharmaceutical compound may be made into an external formulation suitable for topical application to the skin using techniques well known to people skilled in the art. For example, it may be any of the following, but is not limited to: emulsion, gel, ointment, cream, patch, liniment, powder, aerosol, spray, lotion, syrup. lotion, serum, paste, foam, drop, suspension, salve, and bandage.

In some embodiments, the external formulation is made by mixing a pharmaceutical composition with a base known to people skilled in the art.

In some embodiments, the substrate may comprise one or more of the following additives: water, alcohols, glycol, hydrocarbons (such as petroleum, jelly, and white petrolatum), wax (such as paraffin and yellow wax), preserving agents, antioxidants, surfactants, absorption enhancers, stabilizing agents, gelling agents, stabilizing agents, gelling agents (such as Carbopol® 974P, microcrystalline cellulose, and carboxymethylcellulose), active agents, humectants, odor absorbers, fragrances, pH adjusting agents, chelating agents, emulsifiers, occlusive agents, emollients, thickeners, solubilizing agents, penetration enhancers, anti-irritants, colorants, propellants, etc. The selection and quantity of these additives is a matter of professionalism and routine for people skilled in the art.

In some embodiments, the skincare products may contain an acceptable adjuvant that is widely used in the manufacture of the care products. For example, the acceptable adjuvant may include one or more of the following adjuvants: a solvent, a gelling agent, an active agent, a preservative, an antioxidant, a screening agent, a chelating agent, an interface activator, a coloring agent, a thickening agent, a filler, a fragrance, and an odor absorber. The selection and quantity of these reagents can be adjusted to suit the actual requirements.

In some embodiments, the skincare products can be manufactured into a form suitable for skincare or makeup using techniques well known to those having ordinary skill in the art, including, but not limited to, an aqueous solution, an aqueous-alcohol solution or an oily solution, an oil-in-water type, a water-in-oil type or a composite type emulsion gel, ointment, cream, mask, patch, pack, liniment, powder, aerosol, spray, lotion, slurry, past, foam, dispersion, drop, mousse, sunblock, sunscreen essence, sunscreen cream, sunscreen spray, tonic water, foundation, makeup remover product, soap, and other body cleansing product.

In an embodiment of the present invention, cosmetic compositions and personal care products are provided that comprise a pigmented composition capable of exhibiting a color change, such as a pigment grind comprising pigment particulates in a carrier and a styrene-maleic anhydride copolymer. The cosmetic compositions may be in the form of any cosmetic or personal care product to impart color to a human integument. For example, the cosmetic composition may be, without limitation, in the form of lipstick, lip color, lip gloss, nail polish, foundation, face powder, body powder, concealer, blush, eye shadow, eye liner, mascara, or bronzer. The personal care product may be in any suitable form to impart color to a human integument. For example, the personal care product may include day creams or lotions, night creams or lotions, sunscreen lotions, creams, or oils and other SPF products, moisturizers, salves, ointments, gels, body milks, artificial tanning compositions, depilatories, etc.

The cosmetic compositions and personal care products of the invention are applied to the human integumentary system, including, skin, lips, nails, hair, and other keratinous surfaces. As used herein, the term “keratinous surface” refers to keratin-containing portions of the human integumentary system, which includes, but is not limited to, skin, lips, hair (including hair of the scalp, eyelashes, eyebrows, facial hair, and body hair such as hair of the arms, legs, etc.), and nails (toenails, fingernails, cuticles, etc.) of mammalians, preferably humans.

The cosmetics or personal care products capable of exhibiting a color change can be applied to any area of the skin, and preferably on the face, the neck, the hands, the feet, or other areas of the body, such as arms, legs, and back.

In some embodiments, the skin care product could also be used in combination with one or more external use agents selected from the following activities: a whitening agent [such as tretinoin, catechin, citric acid, arbutin and vitamin C], a moisturizer, an anti-inflammatory agent, a bactericide, an ultraviolet absorber, a plant extract [such as aloe extract], a skin nutrient, an anesthetic, an anti-acne agent, an antipruritic, an analgesic, an antidermatitis agent, an antihyperkeratolytic agent, an anti-dry skin agent, an antipsoriatic agent, an antiaging agent, an antiwrinkle agent, an antiseborborheic agent, a wound-healing agent, a corticosteroid, and a hormone. The selection and quantity of these external preparations falls within the professionalism and routine technology of those having ordinary skill in the art.

In some embodiments, the food product can be used as a food additive, added by the conventional method in the preparation of the raw material, or added during the production of the food, and matched with any edible material to be made into food products for human and non-human animals.

In some implementations, the types of the food products include, but are not limited to, beverages, fermented foods, bakery products, health foods, and dietary supplements.

Depending on the desired purpose, the pharmaceutical composition in accordance with the present invention could be provided in any suitable form without special limitations. For example, the pharmaceutical composition could be administered to a subject in need by an oral administration. Depending on the form and purpose, suitable carriers could be chosen and used to provide the pharmaceutical composition, wherein the carriers include excipients, diluents, auxiliaries, stabilizers, absorbent retarders, disintegrating agents, hydrotropic agents, emulsifiers, antioxidants, adhesives, binders, tackifiers, dispersants, suspending agents, lubricants, hygroscopic agents, etc.

The food composition provided according to the invention may be a beverage, a solid food, or a semi-solid food. The food composition may be provided in the form of a health food, a health product, a functional food, a nutritional supplement or a special nutritional food. For example, but not limitation, the food composition may be a dairy product, a processed meat product, a bakery product, a pasta product, a biscuit, an ice product, an oral tablet, a capsule, a fruit juice, a tea, an sparkling water, an alcoholic beverage, a sports drink, a nutritional beverage, a lactation-free food for infants and young children, and the like. Preferably, the food composition is provided in the form of a health food or health product.

In addition, depending on the form of use and the need, any suitable food additive may be included in the food composition provided according to the present disclosure. Examples include, but are not limited to, preservatives, bactericides, antioxidants, bleaching agents, color preservatives, bulking agents, nutritional additives, coloring agents, flavoring agents (e.g. sweeteners), thickening agents, binding agents, chemicals for the food industry, emulsifiers, and agents for quality improvement, brewing and food preparation.

The recommended daily dosage, use standards and use conditions for a specific population (e.g., patients suffering from hyperlipidemia, pregnant woman, etc.), or the recommendations for a use in combination with another food product or medicament could be indicated on the exterior package of the health food, daily supplement, functional food, nutritional supplement, and/or special nutritional food provided in accordance with the present invention. Thus, it is suitable for the user to take the health food, daily supplement, functional food, nutritional supplement, and/or special nutritional food by him- or herself safely and securely without the instructions of a doctor, pharmacist, or related executive.

The experimental steps in the following embodiments are performed at room temperature (25±5° C.) and atmospheric pressure (1 atm) if not otherwise specified.

### [Example 1]: Preparation of Rubus fruticosus Extracts

First, the Rubus fruticosus (from Chile) is pulverized to a size of about 12 mm size, and then the homogenized Rubus fruticosus is extracted with water, alcohols, aqueous alcohols or a combination thereof as an extraction solvent. The extracting is at an extraction temperature ranging from 50° C. to 100° C., preferably 80° C. to 90° C. In the present example, the extraction time ranging from 1:1 to 5, preferably 5:1. In the present example, the extraction time is 0.5˜3 hours, preferably 1 hour.

After cooling down to room temperature, the extract is further centrifuged at 800˜1300 rpm for 5˜10 minutes at 15° C.˜25° C. to obtain a supernatant, which could be filtered through a 400 mesh filter to remove residual solids. The supernatant can be filtered through a 400 mesh filter to remove residual solids. A concentrated product is further concentrated at 55° C. to 65° C. under reduced pressure. In an embodiment, in order to obtain a solid form of Rubus fruticosus extract, the aforementioned reduced pressure concentrated Rubus fruticosus extract is spray dried to remove the solvent, thus obtaining a Rubus fruticosus extract powder. In another embodiment, 2.5 g of the aforementioned concentrated Rubus fruticosus extract is added to 50 mL of water or beverage to obtain a Rubus fruticosus extract beverage having a concentration of 0.05 mg/mL, the Rubus fruticosus extract beverage having a brix level of 5.0±0.3° Bx.

### [Example 2]: Determination of Total Flavonoid and Total Polyphenol Content of the Rubus fruticosus Extract

The analytical method for the determination of total flavonoid content is modified based on the method described in Zhishen Jia et al., (1999), Food Chemistry, 64: 555-559. After mixing well, 200 μL of 5% sodium nitrite is added to stand for 6 minutes. 200 μL of 10% aluminum nitrate is added to stand for another 6 minutes. Finally, 2 mL of 4% sodium hydroxide and 1.4 mL of H₂O are added, mixed well, analyzed at 500 nm, and standard curves are plotted using rutin as the standard. In the present example, the total flavonoid content of the Rubus fruticosus extract of Example 1 ranges from 1000 and 3000 ppm, preferably ranges from 2000 and 3000 ppm, and specifically 2248.9 ppm.

For the total polyphenol content assay, 100 mL of the sample is diluted 10 times with water. 100 μL of the diluted sample is added into a centrifuge tube. Then, 500 μL of Folin-Ciocalteu phenol reagent is added to the centrifuge tube and mixed with the diluted sample to stand for 3 minutes. Then, 400 μL of 7.5% sodium carbonate is added to stand for 30 minutes to obtain the reaction solution to be tested. After the second standing, 200 μL of the reaction solution to be tested is taken into a 96-well plate, and the absorbance value of the reaction solution at 750 nm is measured. Moreover, gallic acid is used as a standard to make the standard curve. In the present example, the total polyphenol content of the Rubus fruticosus extract of Example 1 ranges from 8000 and 11000 ppm, preferably ranges from 9000 and 10000 ppm, and specifically 9364.6 ppm.

### [Example 3]: Cellular Experiment—Inhibition of Reactive Oxygen Species (ROS) Generation by the Rubus fruticosus Extract

In the present example, a fluorescence probe DCFH-DA is used in conjunction with a flow cytometer to determine the changes in the reactive oxygen species content of human skin fibroblasts CCD-966sk after treatment with the Rubus fruticosus extract.

Materials and Instruments:
1. Cell line: Human skin fibroblast CCD-966sk (Bioresource Conservation and Research Center (BCRC), No. 60153), hereafter referred to as CCD-966sk cells.
2. Medium: basal medium containing 10 vol % FBS (fetal bovine serum, purchased from Gibco). The basal medium is made up of Eagle's minimum essential medium (MEM), purchased from Gibco, Product No. 15188-319, with additional ingredients containing 1 mM sodium pyruvate, 1.5 g/kg of FBS, and 1.5 g/kg of FBS. sodium pyruvate (Gibco), 1.5 g/L sodium bicarbonate (Sigma) and 0.1 mM non-essential amino acid solution (Gibco).
3. Phosphate buffer solution (PBS solution): purchased from Gibco, product No. 10437-028.
4. DCFH-DA solution: Dichloro-dihydro-fluorescein diacetate (DCFH-DA; Product No. SI-D 6883, purchased from Sigma) is dissolved in dimethyl sulfoxide (DMSO, purchased from Sigma, Product No. SI-D6883-50 MG) to prepare a 5 mg/mL solution of DCFH-DA.
5. Flow cytometry, Beckman, Catalog No. 660519.
6. Hydrogen peroxide (H₂O₂): purchased from Sigma-Aldrich, product No. 95299-1L.
7. Trypsin (Trypsin-EDTA): 10× Trypsin-EDTA (purchased from Gibco) diluted 10 times with 1×PBS solution.
8. Rubus fruticosus extract: The Rubus fruticosus extract used in the present example is obtained by Example 1 as described hereabove.

Experimental Steps:

The experiment will be divided into an experimental group, a control group (without the addition of the Rubus fruticosus extract and is not treated with hydrogen peroxide), and the control group (without the addition of the Rubus fruticosus extract and is treated with hydrogen peroxide) will be divided into three groups. Each group will be subjected to a duplicate test.
1. Inoculate CCD-966sk cells at 1×10⁵ cells per well in a 6-well culture plate containing 2 mL of medium per well.
2. Incubate the culture plate at 5% CO₂, 37° C. for 24 hours.
3. Remove the medium.
4. Add 2 mL of experimental medium to each well of the culture plate and incubate at 37° C. for 1 hour.
   The experimental medium for the experimental group is 5 μL of the Rubus fruticosus extract of 2 mL cell culture medium (i.e. the weight percentage of the Rubus fruticosus extract in the cell culture medium is 0.25%).

The experimental medium for the control group is a 2 mL of pure cell culture medium (i.e. without the Rubus fruticosus extract).

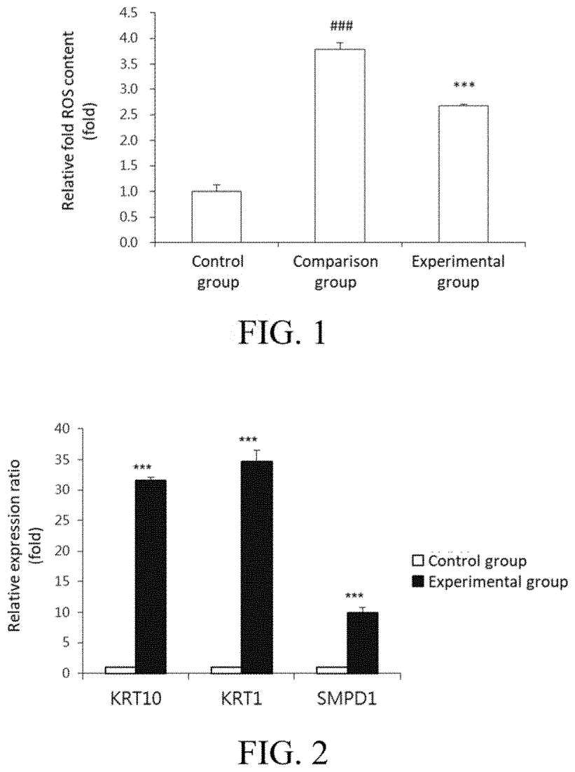

The experimental medium for the comparison group is 2 mL of pure cell culture medium (i.e., without the Rubus fruticosus extract).
5. Add 2 μL of DCFH-DA solution at a concentration of 5 μg/mL to the cell culture medium in each well and treat the cells with DCFH-DA for 15 minutes.
6. After DCFH-DA treatment, H₂O₂ is added to the experimental medium of the experimental group and the experimental medium of the comparison group. The reaction is carried out at 37° C. for 1 hour. Specifically, 35 wt % of hydrogen peroxide is diluted to 100 mM (104 of hydrogen peroxide is added to 990 μL of double-distilled water). Then 20 μL of 100 mM of hydrogen peroxide is added to 2 mL of cell culture plate.
7. After the reaction, each well is washed twice with 1 mL of 1×PBS solution.
8. Add 200 μL of trypsin to each well and react in the dark for 5 minutes. After the reaction, add 6 mL of cell culture medium to terminate the reaction.
9. Collect the cells and cell culture medium in each well into individual 1.5 mL centrifuge tubes and centrifuge the tubes containing the cells and medium at 400×g for 10 minutes.
10. After centrifugation, remove the supernatant and dissolve the cellular precipitate in 1×PBS solution.
11. Centrifuge at 400×g for another 10 minutes.
12. After centrifugation, remove the supernatant and resuspend the cells with 1 mL of 1×PBS solution in dark to obtain the cell solution to be tested.
13. The fluorescence signal of DCFH-DA in the cytosol to be tested in each well is detected using a flow cytometer. DCFH-DA is hydrolyzed into DCFH (dichlorodiohydrofluorophore) and oxidized by reactive oxygen species to DCF (dichlorofluorescein), which emits green fluorescence. The fluorescence intensity of DCFH-DA-treated cells can reflect the content of reactive oxygen species in the cells. The fluorescence intensity of the cells treated with DCFH-DA can reflect the content of reactive oxygen species in the cells. The ratio of the number of cells with high expression of reactive oxygen species to the original number of cells can be obtained. Because the experiment is performed in duplicate, the measurement results of the duplicate experiments in each group are averaged to obtain the average value. The mean value of the relative ROS generation of the control group is 100%. The mean values of the control group and experimental group are converted to the relative ROS generation, as shown in FIG. 1.

Experimental Results:

As shown in FIG. 1, by comparing the results of the control group and the comparison group, the relative ROS production after hydrogen peroxide treatment significantly increased by about 280%, which indicates that hydrogen peroxide treatment does lead to intracellular production of reactive oxygen species and cause subsequent damage to skin fibroblasts. On the other hand, comparing the results of the control group with the results of the experimental group, when the cells are treated with the Rubus fruticosus extract, the relative ROS production is significantly reduced by about 29%, even lower than the ROS production of the control group, which indicates that the Rubus fruticosus extract can effectively reduce the production or accumulation of ROS in the cells. In other words, the Rubus fruticosus extract can be used as a reactive oxygen species scavenger. That is, by reducing an amount of reactive oxygen species in cells, the Rubus fruticosus extract reduces the oxidative damage caused by reactive oxygen species.

### [Example 4] Cell Experiment—the Rubus fruticosus Extract Increases the Expression of Moisturization-Related Genes

In the present example, the RNA extraction kit, reverse transcriptase, and KAPA SYBR® FAST qPCR reagent kit are used in conjunction with a quantitative PCR instrument to determine the changes in the moisturization-related genes in human melanoma cells after treatment with the Rubus fruticosus extract. Please refer to Example 3 for the experimental procedures.

For example, the moisturization-related genes are KRT10 gene (Gene ID:3858), KRT1 gene (Gene ID:3848), and SMPD1 gene (Gene ID:6609).

Keratin 10 (KRT10) and keratin 1 (KRT 1) are responsible for encoding keratin, the major building block of skin. Sphingomyelin phosphodiesterase (SMPD1), tyrosinase-related protein-1 (TYRP1) is responsible for encoding sphingomyelin phosphodiesterase, which converts sphingomyelin to ceramide.

Materials and Instruments:
1. Cell line: Cell line: Human primary epidermal keratinocytes (HPEK-50) (CELLnTEC company (Switzerland), HPEK-50)
2. Culture medium: Keratinocyte-SFM (purchased from Thermo Inc., No. 17005042).
3. RNA extraction kit (purchased from Geneaid ltd., Taiwan, Lot No. FC24015-G).
4. Reverse Transcriptase (SuperScript® III Reverse Transcriptase) (Invitrogen, USA, No. 18080-051).
5. Genetic primers for the measurement markers, which contained the SRD5A1 gene, SRD5A2 gene, AR gene, KROX20 gene, SCF gene, VEGF gene, IGF1 gene, TGF-B gene, and also the internal control group (TBP gene).
6. KAPA SYBR® FAST qPCR reagent kit (purchased from Sigma, USA, No. 38220000000).
7. ABI StepOnePlus™ real-time PCR system (ABI StepOnePlus™ Real-Time PCR system (Thermo Fisher Scientific Inc., USA)).
8. Rubus fruticosus extract: The Rubus fruticosus extract used in the present example is obtained by Example 1 as described hereabove.

Experimental Steps:

First, 1.5×10⁵ human primary skin keratinocytes are incubated in a six-well culture plate containing 2 ml of the above medium per well for 24 hours at 37° C. The human primary skin keratinocytes from each well are divided into a control group and an experimental group (two groups in total) according to the following test conditions.

Test Conditions:

The control group is incubated with human primary skin keratinocytes using 2 mL of pure culture medium, without adding other ingredients. The experimental group is incubated with human primary skin keratinocytes with 2 mL of medium containing a concentration of 0.25 mg/mL of the Rubus fruticosus extract prepared as in the above example for 24 hours. Each group is performed triplicated.

The treated human primary skin keratinocytes (i.e., the control group and the experimental group) are subjected to cell lysis solution to break the cell membrane to form two sets of cell solutions. Then, 1000 ng (ng) of the extracted RNA is used as a template for each group, and the extracted RNA is reverse transcribed by SuperScript® III reverse transcriptase (Invitrogene Inc. USA, No. 18080-051). The extracted RNA is then reverse transcribed to the corresponding c DNA using the ABI StepOnePlus™ Real-Time PCR system (Thermo Fisher Scientific, Inc., USA), KAPA SYBR FAST (Sigma, USA, No. 38220000000) and the primers in Table 1 (SEQ ID NO: 1-8) are used for perform quantitative real-time reverse transcription polymerase chain reaction on the two sets of cDNAs to observe the expression of KRT10 gene, KRT1 gene and SMPD1 gene in two sets of human primary skin keratinocytes. The quantitative real-time reverse transcription polymerase chain reaction is performed at 95° C. for 20 seconds, followed by 95° C. for 3 seconds and 60° C. for 30 seconds, and repeated for 40 cycles. The genes are quantified using the 2-ΔCt method. In this way, the quantitative real-time reverse transcription polymerase chain reaction by cDNA can indirectly quantify the mRNA expression of KRT10, KRT1, and SMPD1 genes. Then, the expression of proteins encoded by KRT10, KRT1, and SMPD1 genes are determined.

TABLE 1

| Primer | | |
|---|---|---|
| names | SEQ ID NOs | Primer sequences |
| KRT10-F | SEQ ID NO: 1 | TCCTACTTGGACAAAGTTCGGG |
| KRT10-R | SEQ ID NO: 2 | CCCCTGATGTGAGTTGCCA |
| KRT1-F | SEQ ID NO: 3 | AGAGTGGACCAACTGAAGAGT |
| KRT1-R | SEQ ID NO: 4 | ATTCTCTGCATTTGTCCGCTT |
| SMPD1-F | SEQ ID NO: 5 | CTGACTCTCGGGTTCTCTGG |
| SMPDI-R | SEQ ID NO: 6 | TCCACCATGTCATCCTCAAA |
| TBP-F | SEQ ID NO: 7 | TATAATCCCAAGCGGTTTGC |
| TBP-R | SEQ ID NO: 8 | GCTGGAAAACCCAACTTCTG |

* F is forward, R is reverse.

In particular, the relative gene expressions of the KRT10, KRT1, and SMPD1 genes described below and shown in the figure are presented as relative fold, where the standard deviation is calculated using the STDEV formula in Excel software and is analyzed for statistically significant differences using a one-tailed Student t-test in Excel software. In the figure, The “*” means p-value is less than 0.05, “*” means p-value is less than 0.01, and “***” means p-value is less than 0.001. The more “*” means the more statistically significant difference.

Please refer to FIG. 2. The expression of TBP gene in the control group is considered as 1-fold. The expression of KRT10 gene in the experimental group is 31.6-fold compared with the control group. The expression of KRT1 gene in the experimental group is 34.7-fold compared with the control group. The expression of SMPD1 gene in the experimental group is 9.9-fold compared with the control group. It can be seen that when human melanoma cells are treated with 0.25 mg/mL of the Rubus fruticosus extract, the KRT10 and KRT1 genes are positively correlated with the formation of keratin. Keratin contains natural moisturizing factor NMF, which reduces the intercellular space and protects the dermis from external stimuli, skin damage, and inflammation. As shown in FIG. 2, the present disclosure can enhance the genetic expression of the moisturization-related genes (KRT10 gene, KRT1 gene, and SMPD1 gene) of the cells. Therefore, the Rubus fruticosus extract possesses moisturizing effect on the skin.

### [Example 5] Cellular Experiment—Rubus fruticosus Extract Reducing the Expression of Melanin Genes

In the present example, RNA extraction kit, reverse transcriptase, KAPA SYBR® FAST qPCR kit, and quantitative PCR instrument are used for determine the changes of whitening-related genes in human melanoma cells after treatment with the Rubus fruticosus extract.

For example, the melanin-related genes are TYR gene (Gene ID: 7299), TYRP1 gene (Gene ID:7306), MITF gene (Gene ID:4286), and MCIR gene (Gene ID:4157).

Microphthalamia-associated transcription factor (MITF) is a transcription factor required for the development of normal melanocytes in the skin. It regulates the gene expression of several other proteins involved in melanin production, such as tyrosinase (TYR), tyrosinase-related protein-1 (TYRP1), etc. Melanocortin 1-receptor (MC1R) gene is a key gene in the process of melanin synthesis in human skin.

Therefore, the TYR gene, TYRP1 gene, MITF gene, and MC1R gene are used as the markers of analysis in the present example.

Materials and Instruments:
1. Cell line: human melanoma cell A375.S2 (ATCC, No. CRL-1872).
2. Medium: MEM-NAA medium containing 10% fetal bovine serum (FBS) (GIBCO, No. 10438-026, USA), 1% Antibiotic-Antimycotic (Gibco, No. 15240-062), 1 mM sodium pyruvate (Gibco, No. 11360-070) in MEM-NAA medium (Gibco, No. 41500-03).
3. RNA extraction kit (purchased from Geneaid, Taiwan, Lot No. FC24015-G).
4. Reverse Transcriptase (Super Script® III Reverse Transcriptase) (Invitrogen, USA, No. 18080-051).
5. Measurement of the subject gene primers containing the TYRP1 gene, the TYR gene, the MITF gene and the MC1R gene, plus the internal control group (GAPDH gene).
6. KAPA SYBR® FAST q PCR reagent set (purchased from Sigma, USA, No. 38220000000).
7. ABI Step One Plus™ Real-Time PCR system (ABI Step One Plus™ Real-Time PCR system (Thermo Fisher Scientific, Inc., USA)).
8. Rubus fruticosus extract: The Rubus fruticosus extract used in the present example is obtained by Example 1 as described above.

Experimental Steps:

First, 1.5×10⁵ human melanoma cells are incubated in a six-well culture plate containing 2 ml of the above medium per well for 24 hours at 37° C. The human melanoma cells from each well are divided into a control group and an experimental group (two groups in total) according to the following test conditions.

Test Conditions:

The experimental control is prepared using 2 mL of culture medium alone to culture human melanoma cells without adding other components. The experimental group is prepared as in the above example by incubating the Rubus fruticosus extract. The human melanoma cells are incubated for 24 hours in 2 mL medium containing a concentration of 0.125 mg/mL of the Rubus fruticosus extract as prepared in the above example.

The treated human melanoma cells (i. e., control and experimental groups) are subjected to cell lysis solution to break the cell membrane to form two sets of cell solutions. Then, 1000 nanogram (ng) of the extracted RNA is used as template for each group, and the extracted RNA is reverse transcribed by Super Script® III enzyme (Invitrogene, Inc., USA, Lot No. 18080-051). The extracted RNA is then reverse transcribed to the corresponding cDNA using the ABI StepOnePlus™ Real-Time PCR system (Thermo Fisher Scientific, Inc., USA), KAPA SYBR FAST (Sigma, USA, No. 38220000000) and the primers in Table 1 (SEQ ID NO: 9-18) are used for perform quantitative real-time reverse transcription polymerase chain reaction on the two sets of cDNAs to observe the expression of KRT10 gene, KRT1 gene and SMPD1 gene in two sets of human primary skin keratinocytes. The quantitative real-time reverse transcription polymerase chain reaction is performed at 95° C. for 20 seconds, followed by 95° C. for 3 seconds and 60° C. for 30 seconds, and repeated for 40 cycles. The genes are quantified using the 2-ΔCt method. In this way, the quantitative real-time reverse transcription polymerase chain reaction by cDNA can indirectly quantify the mRNA expression of KRT10, KRT1, and SMPD1 genes. Then, the expression of proteins encoded by KRT10, KRT1, and SMPD1 genes are determined.

TABLE 2

| Primer | | |
|---|---|---|
| names | SEQ ID NOs | Primer sequences |
| TYRP1-F | SEQ ID NO: 9 | GACACGCCTCCTTTTTATTC |
| | | CA |
| TYRP1-R | SEQ ID NO: 10 | ATGGGTTTGTCCCCCTGTTC |
| TYR-F | SEQ ID NO: 11 | CTCAAAGCAGCATGCACAAT |
| TYR-R | SEQ ID NO: 12 | GCCCAGATCTTTGGATGAAA |
| MITF-F | SEQ ID NO: 13 | GCCTCCAAGCCTCCGATAAG |
| MTTF-R | SEQ ID NO: 14 | GCACTCTCTGTTGCATGAAC |
| | | T |
| MC1R-F | SEQ ID NO: 15 | CATCATCGACCCCCTCATCT |
| | | AC |
| MC1R-R | SEQ ID NO: 16 | CAGGAACCAGACCACACAAT |
| | | ATCA |
| GAPDH-F | SEQ ID NO: 17 | CTGGGCTACACTGAGCACC |
| GAPDH-R | SEQ ID NO: 18 | AAGTGGTCGTTGAGGGCAAT |
| | | G |

* F is forward, R is reverse.

In particular, the relative gene expressions of TYR, TYRP1, MITF, and MC1R genes shown in the figures below are presented as relative fold, where the standard deviation is calculated using the STDEV formula in Excel software and is analyzed for statistically significant differences using a one-tailed Student t-test in Excel software. In the figure, The “*” means p-value is less than 0.05, “*” means p-value is less than 0.01, and “***” means p-value is less than 0.001. The more “*” means the more statistically significant difference.

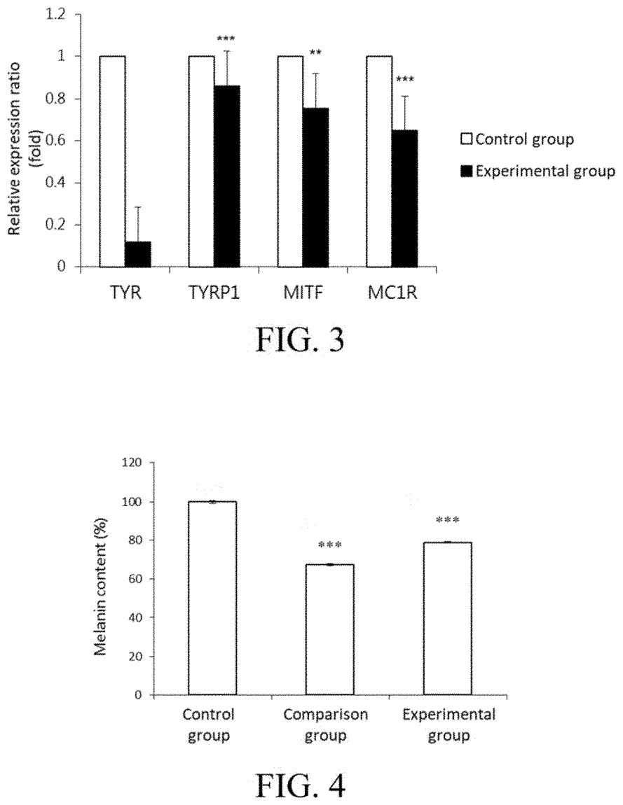

Please refer to FIG. 3. the expression of the GAPDH gene in the control group is I-fold. The expression of the TYR gene in the experimental group is 0.12-fold compared with the control group. The expression of the TYRP1 gene in the experimental group is 0.86-fold compared with the control group. The expression of the MITF gene in the experimental group is 0.75-fold compared with the control group. The expression of the MC1R gene in the experimental group is 0.65-fold compared with the control group. The results represent an 88% reduction in the expression of the TYR gene in the experimental group compared to the control group, a 14% reduction in the expression of the TYRP1 gene in the experimental group compared to the control group, a 25% reduction in the expression of the MITF gene in the experimental group compared to the control group, and a 35% reduction in the expression of the MC1R gene in the experimental group compared to the control group. It can be seen that after human melanoma cells are treated with 0.125 mg/mL of the Rubus fruticosus extract, the MC1R and MITF genes are positively correlated with melanin formation. The results shown in FIG. 3 indicate that the Rubus fruticosus extract of the present disclosure inhibits the gene expression of TYR gene, TYRP 1 gene, MC1R gene, and MITF gene in melanoma cells. Therefore, the Rubus fruticosus extract possesses an inhibitory effect on melanin formation.

### [Example 6] Cellular Experiment—Rubus fruticosus Extract Reduces Melanin Production

The changes in melanin content of melanoma cell line B16F10 after being treated with Rubus fruticosus extract are determined by an ELISA reader (enzyme-linked immunosorbent assay reader).

Materials and Instruments:
1. Cell line: Mouse melanoma cell B16F10, purchased from American Type Culture Collection (ATCC®, No. 6475), hereinafter referred to as B16F10 cells.
2. Medium: Dulbecco's modified minimal essential medium (DMEM, purchased from Gibco, Cat. 12100-038) is supplemented with additional ingredients to contain 1 vol % antibiotic solution (Antibiotic Antimycotic Solution, purchased from Gibco, 15240-062) and 10 vol % FBS (fetal bovine Serum, purchased from Gibco, 10437-028). Gibco, 15240-062).
3. Phosphate Buffer Solution (PBS solution): purchased from Gibco, Product No. 10437-028.
4. Prepare a solution of 1N NaOH (Sigma, Product No. 221465) in secondary distilled water.
5. ELISA reader (purchased from BioTek, product number FLx 800).
6. Rubus fruticosus extract: The Rubus fruticosus extract used in the present example is obtained by Example 1 as described hereabove.

Experimental Steps:

The experiment will be divided into three groups: experimental group, control group and comparison group, and each group are performed triplicate.
1. Inoculate B16F10 cells at 1.5×10⁵ cells per well in a 6-well culture plate containing 3 mL of medium per well.
2. Incubate the culture plates in 5% CO₂ at 37° C. for 24 hours.
3. Then, without disturbing the attached cells, remove the medium from each well.
4. The experiment is divided into control group, comparison group, and experimental group. In each group, 2 mL of fresh DMEM culture medium is added. In the control group, additional Kojic acid is added to the culture medium at a concentration of 0.25 mg/mL. Kojic acid is widely recognized as a substance with melanin production-reducing effect. In the experimental group, additional Kojic acid is added to the culture medium at a concentration of 0.25 mg/mL of Rubus fruticosus extracts. Each group is performed triplicated and incubated at 37° C. for 24 hours.
5. The completed experimental group and the comparison group are transferred to a blue light chamber and exposed to blue light at room temperature (25±5° C.) for 3 hours. In addition, the control group is moved to a dark room and allowed to stand at room temperature for 3 hours.
6. After the reaction, the cells are incubated at 37° C. for 48 hours.
7. Afterwards, remove the medium and wash the cells twice with PBS solution.
8. 200 μl of Trypsin (Trypsin-EDTA (10×), purchased from Gibco; Product No. 15400-054) is added to each well for 3 minutes. After the reaction is completed, 6 mL of medium solution is added to terminate the reaction. The suspended cells and medium are collected from each well into a corresponding 15 mL centrifuge tube, and each tube is centrifuged at 400×g for 5 minutes to allow the cells to precipitate.
9. Wash the precipitated cells twice with PBS solution and then resuspend the cells with 200 μL of PBS solution.
10. Freeze the cell suspension in liquid nitrogen for 10 minutes, then leave the cell suspension at room temperature for about 30 minutes until it is completely thawed.
11. After thawing is completed, the tube at 12,000 g is centrifuged for 3 minutes.
12. Remove the supernatant and re-suspend the cell precipitate with 120 μL of 1N sodium hydroxide solution and allow the tube to dry-bath at 60° C. for 1 hour to obtain the sample to be tested.
13. Transfer 100 μL of the sample to be tested into a 96-well plate and measure the absorbance value of the cell solution at 450 nm using an ELISA reader.

Experimental Results:

The relative melanin expressions of the experimental and control groups are calculated according to the following formula: Relative melanin expression (%)=(OD 450 value of each group/OD450 value of the control group)×100%. Since the experiment is conducted in triplicate, the results of the triplicate experiment are averaged and shown in FIG. 4.

As shown in FIG. 4, the effect of reducing melanin production can be achieved by the treatment of the Rubus fruticosus extract of the present disclosure. Specifically, the melanin production of the experimental group (the Rubus fruticosus extract) is 78.81% compared with the control group, and the melanin production of the control group is 67.42% compared with the control group. Compared with the control group, the melanin production of the experimental group was reduced by about 21.19%, and the effect of the Rubus fruticosus extract of the present disclosure was similar to the effect of kojic acid in reducing melanin production. Therefore, the Rubus fruticosus extract prepared by the embodiment of the present disclosure can effectively reduce melanin production by melanocytes. Therefore, the Rubus fruticosus extract can be used as a component of relevant composition for reducing skin dark spots, enhancing skin brightness, and skin whitening.

As shown in the above cell experiments, the Rubus fruticosus extract can reduce the production of reactive oxygen species (ROS) in skin fibroblasts and prevent skin damage caused by UV rays. The Rubus fruticosus extract can reduce the amount of melanin-related genes (TYRP1 gene, MC1R gene, and MITF gene) and inhibit melanin production, reducing the dark spots caused by blue light, and maintaining clear skin.

In order to confirm the efficacy of the Rubus fruticosus extract on humans, the following human trials have been conducted.

### [Example 7] Detection of an Amount of UV Red Spot (UV Spot) in Skin

UV light from the VISIA complexion analysis system (Canfield scientific, USA) is used for photograph the skin of the face.

UV light is absorbed by melanin, increasing the visibility of pigmentation and detecting melanin spots in the epidermis that are not visible to the naked eye. The higher the measurement value, the more UV pigmentation there is.

A beverages of the Rubus fruticosus extract of the present disclosure is prepared with water to provide a suitable effective dose to the subjects. In the present example, 7 subjects (male and female aged 2065) intake a bottle of the prepared beverage at a concentration of 0.005 mg/mL of the Rubus fruticosus extract (50 mL) for a total of 8 weeks. The number and area of red spots on the whole face of the subjects' cleaned facial skin are measured with a full-face skin detector (7th Generation VISIA Complexion Analysis System; Canfield, USA) before the start of the experiment (week 0, not yet intake), at week 4, and at week 8.

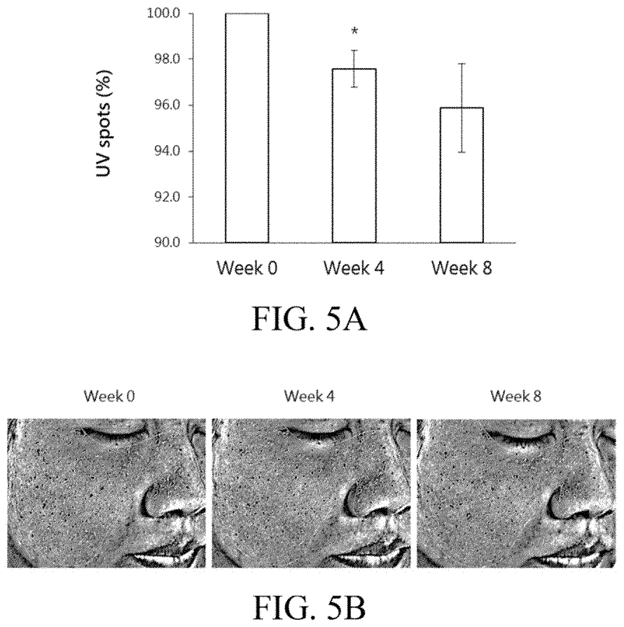

As shown in FIG. 5A, when the subjects took the Rubus fruticosus extract beverages for 4 weeks, the number/area of red spots begin to reduce. After 4 weeks of continuous intake, the number of red spots on the facial skin reduces 2.4% compared with the original number. After 8 weeks of continuous intake, the number of red spots on the facial skin reduces 4.1% compared with the original number. It can also be found in the actual photograph of FIG. 5B that as the period of intakes of the Rubus fruticosus extract beverages increases, the number and area of red spots on the skin are significantly reduced. The number of improved people reaches 86%. The Rubus fruticosus extract possesses the effect of inhibiting the spots formation.

### [Example 8] Measurement of Relative Erythema Index of Skin Redness

The facial skin of the subjects in Example 7 above is photographed using the RBX polarized light technology of the VISIA Complexion Analysis System (Canfield scientific, USA) to detect deep skin vessels or erythema. The higher the measurement, the more severe skin redness.

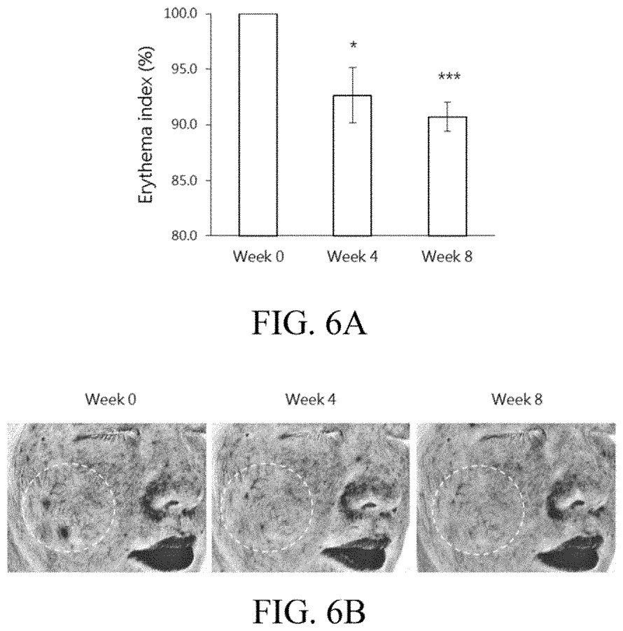

As shown in the FIG. 6A, when the subjects took the Rubus fruticosus extract beverages for 4 weeks, the amount of erythema index begin to reduce. After 4 weeks of continuous intake, the amount of erythema index on the facial skin reduces 7.4% compared with the original number. After 8 weeks of continuous intake, the amount of erythema index on the facial skin reduces 9.3% compared with the original number. It can also be found in the actual photograph of FIG. 6B that as the period of intakes of the Rubus fruticosus extract beverages increases, the amount of erythema index on the skin are significantly reduced. The number of improved people reaches 100%. The results show that the Rubus fruticosus extract beverages of the present disclosure can effectively improve the skin redness of an individual, maintain a stable and healthy condition of the skin. The Rubus fruticosus extract possess a good relieving effect on skin.

### [Example 9] Skin Brightness Detection

The skin brightness measuring probe Glossymeter GL200 (C+K Multi Probe Adapter System, Germany) is reflected by the direct and scattered reflection of light shining onto the skin surface of the subjects in Example 7 above. The higher the measurement value, the more brightness the skin is.

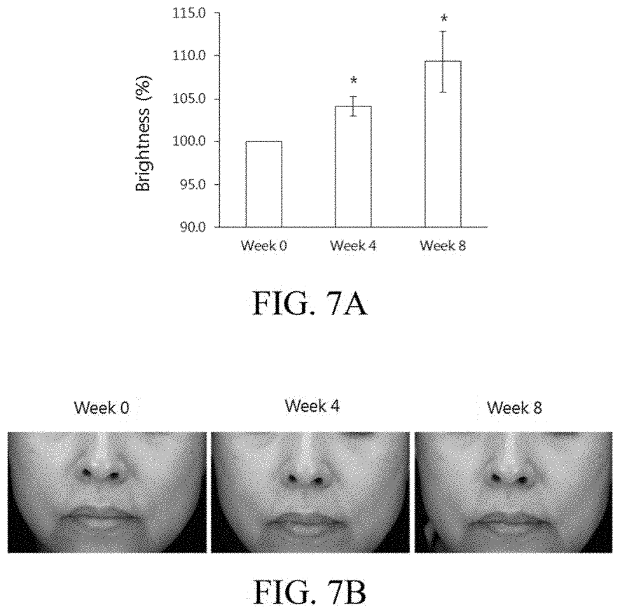

As shown in the FIG. 7A, when the subjects took the Rubus fruticosus extract beverages for 4 weeks, the brightness begin to reduce. After 4 weeks of continuous intake, the brightness on the facial skin reduces 4.2% compared with the original number. After 8 weeks of continuous intake, the brightness on the facial skin reduces 9.4% compared with the original number. It can also be found in the actual photograph of FIG. 7B that as the period of intakes of the Rubus fruticosus extract beverages increases, the brightness on the skin are significantly reduced. The number of improved people reaches 86%. The Rubus fruticosus extract possesses the effect of increasing skin brightness.

In summary, the present disclosure not only proved that the Rubus fruticosus extract of any embodiment inhibits the production of reactive oxygen species, inhibits melanin production by reducing the expression of melanin-related genes (e.g., tyrosinase-related protein-1 (TYRP1), tyrosinase (TYR), microphthalmia-associated transcription factor (MITE), melanocortin receptor 1 (MC1R)) in cells. Therefore, the Rubus fruticosus extract is effective for preventing skin aging phenomena caused by UV rays, relieving skin redness, increasing skin brightness, and increasing skin moisturizing ability. Accordingly, the Rubus fruticosus extract provides overall antioxidant, whitening, and brightness on skin. The Rubus fruticosus extract also protects the skin from UV damage and prevent skin aging.

What is claimed is:

1. A method of manufacturing a skin conditioning composition with Rubus fruticosus extract, wherein the Rubus fruticosus extract is extracted with a solvent containing water.

2. According to the method of claim 1, wherein a total polyphenol content of the Rubus fruticosus extract ranges from 8000 and 11000 ppm.

3. According to the method of claim 1, which is for reducing oxidative damage caused by reactive oxygen species in cells.

4. According to the method of claim 1, which is for reducing a hematocrit level of the skin.

5. According to the method of claim 1, which is for inhibiting melanin production.

6. According to the method of claim 5, wherein the composition inhibits melanin production by reducing an amount of melanin-related genes expressions in cells.

7. According to the method of claim 6, wherein the melanin-related genes comprises: tyrosinase-related protein-1 (TYRP1), tyrosinase (TYR), microphthalmia-associated transcription factor (MITF), and melanocortin receptor 1 (MC1R).

8. According to the method of claim 1, which is for reducing skin redness.

9. According to the method of claim 1, which is for enhancing a moisturizing ability of the skin.

10. According to the method of claim 9, wherein the composition enhances moisturizing ability of the skin by increasing an amount of moisturization-related genes expressions in the cells.

11. According to the method of claim 1, which is for manufacturing a nutraceutical composition, a health product composition, a cosmetic composition, or a topical skin application.
